(19) United States
(12) Patent Application Publication
(10) Pub. No.: US 2024/0352158 A1
(43) Pub. Date: Oct. 24, 2024

(54) CROSSLINKED HYALURONIC ACID PRECIPITATES

(71) Applicant: Prohibix LLC, Philadelphia, PA (US)

(72) Inventor: Brendan Purcell, Boulder, CO (US)

(73) Assignee: Prohibix LLC, Philadelphia, PA (US)

(21) Appl. No.: 18/701,138

(22) PCT Filed: Oct. 17, 2022
(86) PCT No.: PCT/US2022/046831

§ 371 Date: Apr. 12, 2024

Related U.S. Application Data
(60) Provisional application No. 63/355,884, filed on Jun. 27, 2022.
(60) Provisional application No. 63/256,108, filed on Oct. 15, 2021.

(51) Int. Cl. C08B 37/08; A61K 31/728

(52) U.S. Cl. CPC C08B 37/0072; A61K 31/728

(57) ABSTRACT

Hyaluronic acid compositions comprised of precipitated and stabilized hyaluronic acid are disclosed. The compositions may enable higher doses and half-lives of hyaluronic acid delivered in tissues throughout the body to prolong therapeutic effects through controlled solubilization of hyaluronic acid. Compositions may include networks of hyaluronic acid (HA). The networks may provide enhanced control of HA hydration and swelling to increase the amount of exogenous HA that can be delivered into the body and extend the duration of exogenously delivered HA in the body.

### CROSS REFERENCE TO RELATED APPLICATIONS

This application claims the benefit of U.S. provisional application No. 63/256,108, filed Oct. 15, 2021 and titled HIGH DENSITY HYDROLYZING HYALURONIC ACID PARTICLES, and 63/355,884, filed Jun. 27, 2022 and titled CROSSLINKED HYALURONIC ACID PRECIPITATES, both of which are incorporated herein by reference as if fully set forth.

### GOVERNMENT SUPPORT STATEMENT

This invention was made with government support under grant number R44HL140645 awarded by the National Institutes of Health National Heart, Lung, and Blood Institute. The government has certain rights in the invention.

### FIELD

This relates to compositions of hyaluronic acid for local administration in the body.

### BACKGROUND

Hyaluronic acid (HA) is a linear glycosaminoglycan composed of repeating D-glucuronic acid and N-acetyl-D-glucosamine disaccharides. It is a hydrophilic, water-swollen polymer, and is abundant in the extracellular matrix of tissues throughout the body. HA swelling has important mechanical and biological roles in regulating tissue hydration and mechanics, tissue formation, wound healing, and inflammation. HA polymers can be found in the body at molecular weights ranging from 5,000 daltons (Da) to 20,000,000 Da. HA dissolved in aqueous buffers form shear-thinning solutions with viscoelastic properties that are directly proportional to HA concentration and molecular weight. See Snetkov, P., et al., Hyaluronic Acid: The Influence of Molecular Weight on Structural, Physical, Physico-Chemical, and Degradable Properties of Biopolymer. Polymers (Basel), 2020. 12 (8), which is incorporated herein by reference as if fully set forth.

Crosslinks can be introduced between dissolved HA polymers at low extents of crosslinking to increase effective molecular weight or at higher extents of crosslinking to form hydrogels for increased stability in the body. See Burdick, J. A. and G. D. Prestwich, Hyaluronic acid hydrogels for biomedical applications. Adv Mater, 2011. 23 (12): p. H41-56, which is incorporated herein by reference as if fully set forth. HA hydrogels are typically fabricated by first dissolving HA or chemically modified HA in aqueous buffer and then initiating crosslinking through the addition of crosslinking molecules, chemical initiators or through mixing complimentary chemically modified HA. The crosslinked hydrogels swell when placed in aqueous buffer, and hydrogel swelling is inversely proportional to crosslink density. In addition, hydrogel mechanics and stability are directly proportional to crosslink density. The hydrophilic nature of HA is important for its mechanic roles in tissues throughout the body, but also limits the concentration of HA during the crosslinking reaction and, therefore, high extents of chemical modification are typically required to form an insoluble material.

### SUMMARY

In an aspect, the invention relates to a composition comprising a crosslinked precipitate of chemically modified hyaluronic acid.

In an aspect, the invention relates to a method of making a network of hyaluronic acid comprising precipitating a first chemically modified hyaluronic acid to facilitate crosslinking.

In an aspect, the invention relates to a method of treatment comprising injecting into a body of a subject a composition comprising a crosslinked precipitate of chemically modified hyaluronic acid, or the product of precipitating a chemically modified hyaluronic acid.

In an aspect, the invention relates to a composition comprising a stabilized precipitate of hyaluronic acid, wherein the precipitate is stabilized with crosslinks containing hydrolytically degradable chemistries.

In an aspect, the invention relates to composition comprising hyaluronic acid crosslinked in a collapsed conformation to limit hydration and swelling.

In an aspect, the invention relates to hyaluronic acid microparticles that hydrate and swell over time as crosslinks hydrolyze.

In an aspect, the invention relates to a method of forming crosslinked hyaluronic acid materials by precipitating chemically modified hyaluronic acid polymers, wherein the chemical modifications crosslink during or after precipitation.

In an aspect the invention relates to a use of a composition comprising a crosslinked precipitate of chemically modified hyaluronic acid, or the product of precipitating a chemically modified hyaluronic acid reconstituted in aqueous buffer for treating inflammation, treating osteoarthritis, treating ocular disease, treating cardiovascular disease, treating disc degeneration, treating diabetic ulcers, treating pain, reducing inflammation following surgery, improving tissue healing following surgery or a traumatic injury.

### BRIEF DESCRIPTION OF THE DRAWINGS

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIGS. 1A-1F illustrate hydroxyethylmethacrylate modified HA crosslinked with dithiothreitol after precipitation in ethanol.

FIG. 1A shows hydrated particles were imaged with an optical microscope after staining with dimethylmethylene blue. Average diameters (D) and aspect ratios (AR) were measured for at least 100 particles (mean +/− standard deviation). Storage modulus (G′) and loss modulus (G″) of 20 mg/ml particles in PBS were measured with an oscillatory rheometer (TA Instruments HR2, 20 mm cone and plate, 1 degree cone, 28 micron gap).

FIG. 1B shows the opacity of a 20 mg/ml suspensions of precipitated and crosslinked HA was measured with absorbance using a plate reader (Biotek). A 2 wt % solution of 700 kDa sodium hyaluronate in PBS is shown for comparison.

FIG. 1C shows the absorbance at 300 nm of 20 mg/ml suspensions of precipitated and crosslinked HA incubated in PBS at 37° C. measured over time: Day 0 (left), Day 14 (middle), and Day 28 (right) for each of NaHy, 3.7% mod, 7.3% mod, and 15% mod.

FIG. 1D shows the Tan Delta of 20 mg/ml suspensions of precipitated and crosslinked HA incubated in PBS at 37° C. measured over time: Day 0 (left), Day 14 (middle), and Day 28 (right) for each of NaHy, 3.7% mod, 7.3% mod, and 15% mod.and the opacity and rheological properties were measured over time.

FIG. 1E shows the storage modulus (Pa) of 20 mg/ml suspensions of precipitated and crosslinked HA incubated in PBS at 37° C. measured over time: Day 0 (left), Day 14 (middle), and Day 28 (right) for each of NaHy, 3.7% mod, 7.3% mod, and 15% mod.

FIG. 1F shows the loss modulus (Pa) of 20 mg/ml suspensions of precipitated and crosslinked HA incubated in PBS at 37° C. measured over time: Day 0 (left), Day 14 (middle), and Day 28 (right) for each of NaHy, 3.7% mod, 7.3% mod, and 15% mod.

FIG. 2 show acrylate modified HA crosslinked with dithiothreitol after precipitation in ethanol. Hydrated particles were imaged with an optical microscope after staining with dimethylmethylene blue. Scale bar=1 mm. Percent acrylate modification (Mod) was measured using 1H NMR for modification values greater than 2% and extrapolated based on synthesis parameters for values less than 2%. Measured tan delta (0.5Hz frequency, 1% strain, 28 micron gap, 37° C.) of 20 mg/mL HA particles suspended in PBS after 24 hrs swelling. Measured optical absorbance (Abs, 300 nm) of 4 mg/mL HA particles suspended in PBS after 24 hrs swelling.

FIG. 3 shows measured tan delta values of acrylate-dithiothreitol crosslinked HA precipitates over time with incubation in PBS at 37° C. (20 mg/mL, 0.5 Hz frequency, 1% strain, 28 micron gap, 37° C.). X, 6.2% mod; open diamond, 4.0%; closed diamond 2.4%; open square, ˜1.7%; closed square, ˜1.0%, open triangle, ˜0.5%; closed triangle, ˜0.35%, open circle, ˜0.2%, and closed circle, ˜0.1%.

FIG. 4 shows measured optical absorbance values of acrylate-dithiothreitol crosslinked HA precipitates over time with incubation in PBS at 37° C. (4 mg/mL, 300 nm). X, 6.2% mod; open diamond, 4.0%; closed diamond 2.4%; open square, ˜1.7%; closed square, ˜1.0%, open triangle, ˜0.5%; closed triangle, ˜0.35%, open circle, ˜0.2%, and closed circle, ˜0.1%.

FIGS. 5A, 5B, and 5C show viscosity measurements of completely dissolved acrylate-dithiothreitol crosslinked HA precipitates on Day 42 as evidenced by optical clarity and steady tan delta measurements. FIG. 5A shows the results for 0.35% mod, FIG. 5B for 0.2% mod, and FIG. 5C for 0.1% mod.

FIG. 6 shows compressive moduli of acrylate modified HA hydrogels crosslinked with dithiothreitol in either PBS (right bar for each %) or 0.1M phosphate with 50 mM triethanolamine (TEOA, left bar for each %) buffers (n=3 gels per formulation).

FIG. 7 shows swelling ratios of 20 mg/ml acrylate-dithiothreitol crosslinked HA hydrogels over time. The hydrogels were completely dissolved after the final swelling ratio data point (n=3 hydrogels per formulation). Open circle, 2.5% mod; closed circle, 4%; open square, 5%; closed square, 9%; open triangle, 13%; and closed triangle, 20%.

FIGS. 8A and 8B show BSA encapsulation in acrylate-dithiothreitol crosslinked HA precipitates. BSA was loaded at 0:1, 1:19 and 1:4 BSA: HA mass ratios prior to HA precipitation. 10 mg of purified HA/BSA precipitates were reconstituted in 1 mL PBS and BSA released into the supernatant was measured. FIG. 8A illustrates release over the first 24 hrs. Open square, 1:3 BSA: HA; closed circle, 1:19 BSA: HA; and closed triangle, 0:1 BSA: HA. Encapsulated BSA remaining after 24 hrs was measured after hydrolyzing the precipitates in 25 mM NaOH. FIG. 8B illustrates the total recovered BSA (released plus remaining encapsulated).

### DETAILED DESCRIPTION

Certain terminology is used in the following description for convenience only and is not limiting. The words “right,” “left,” “top,” and “bottom” designate directions in the drawings to which reference is made.

“Network” is a collection of polymers in which all polymers are interconnected with each other through physical or chemical crosslinks.

“Hydrogel” is a water swollen network of polymers.

“Crosslink” refers to covalent or non-covalent bonds formed between polymers.

“Crosslinker” refers to molecules that form bonds between polymers. Crosslinkers can react with polymer to form a crosslinking bond or be chemically modified onto the polymer prior to crosslinking to other polymers.

“Hydrolytically degradable” refers to a crosslink that contains covalent bonds that dissociate due to nucleophilic attack of a water molecule. The dissociation may be catalyzed in the presence of an enzyme.

“Extent of chemical modification” refers to the percent of repeat

units of a polymer that are modified with a new chemical group. For HA, the repeat unit is the disaccharide composed of D-glucuronic acid and N-acetyl-D-glucosamine, linked via alternating β-(1→4) and β-(1→3) glycosidic bonds.

“Extent of crosslinking” refers to the percent of repeat units on a polymer that are bound to another polymer or linker molecule through covalent or non-covalent interactions. For HA, the repeat unit is the disaccharide composed of D-glucuronic acid and N-acetyl-D-glucosamine, linked via alternating β-(1→4) and β-(1→3) glycosidic bonds.

“Fraction” is a subset of HA polymers that can be identified by differences in molecular weight, extent or type of chemical crosslinking, extent or type of chemical modification or crosslink density. These subsets may have a distribution around these properties but can be identified through a non-zero difference in the means of their distributions. These subsets may be produced separately through different chemical synthesis or formulation process steps.

“Molecular weight” is a measure of the size of a polymer. As used herein, the term “molecular weight” refers to the number average molecular weight of a polymer as determined by gel permeation chromatography in aqueous buffer. When used to describe an HA polymer, molecular weight refers to the linear uncrosslinked HA.

“Functionality” refers to the number of chemical groups per molecule that are capable of crosslinking to another molecule.

“Storage modulus” or (G′) is a measure of stored energy under force and represents the elastic portion of the complex modulus of viscoelastic materials. G′ can be determined by oscillatory rheology.

“Loss modulus” or (G″) is a measure of energy dissipation under force and represents the viscous portion of the complex modulus of viscoelastic materials. G″ can be determined by oscillatory rheology.

“Physiological buffer” is an aqueous buffer with a pH and ionic concentrations found in the body. Phosphate buffered saline (PBS) which comprises 137 mM NaCl, 2.7 mM KCl, 10 mM Na2HPO4, 1.8 mM KH2PO4, pH 7.4 is a commonly used physiological buffer.

Solubilize refers to the process of an insoluble material going into solution or dissolving into the surrounding liquid.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C” or “A, B, and C,” means any individual one of A, B or C as well as any combination thereof.

Embodiments comprise compositions comprising networks of hyaluronic acid (HA). The networks may provide enhanced control of HA hydration and swelling to increase the amount of exogenous HA that can be delivered into the body and extend the duration of exogenously delivered HA in the body.

An embodiment comprises a composition comprising a crosslinked precipitate of chemically modified hyaluronic acid. The precipitate may be optically opaque. As used herein, “optically opaque” means that the material absorbs or reflects light so as to appear white or turbid in an aqueous buffer. Solutions of HA dissolved in aqueous buffer and water-swollen hydrogels of HA transmit light and appear clear.

The precipitate may comprises microparticles having a between 10 microns and 1 millimeter in diameter when reconstituted in aqueous buffer. The microparticle diameter may be selected from those described below. The precipitate may be stabilized with hydrolytically degradable crosslinks. The precipitate may be stabilized with ester bonds. A protein may be encapsulated in the precipitate.

An embodiment comprises a composition comprising: a network of hyaluronic acid crosslinked with ester bonds. The extent of chemical crosslinking may be less than 2 percent of repeat disaccharides.

An embodiment comprises a method of making a network of hyaluronic acid. The method may comprise precipitating a chemically modified hyaluronic acid. The precipitating may facilitate crosslinking. Two complimentary chemically modified hyaluronic acids may be precipitated together to facilitate crosslinking. The method may further comprise adding chemical initiators to the precipitated hyaluronic acid. The chemical initiators may initiate crosslinking. The method may comprise adding at least one of light or heat to initiate crosslinking. The initiation of crosslinking may be conducted following precipitation. In an embodiment, the hyaluronic acid is chemically modified. The chemical modification may be with methacrylate or acrylate groups. The method may further comprise reconstituting the products in aqueous buffer.

A method may comprise injecting the reconstituted products of a method of making herein into a body of a subject. The subject may be a human. The subject may be a mammal. The injecting into a body may comprise injecting into the synovial fluid to treat osteoarthritis. The injecting into a body may comprise injecting into the pericardial fluid to treat heart disease.

A method may comprise injecting reconstituted composition comprising a crosslinked precipitate of chemically modified hyaluronic acid herein into a body of a subject. The subject may be a human. The subject may be a mammal. The injecting into a body may comprise injecting into the synovial fluid to treat osteoarthritis. The injecting into a body may comprise injecting into the pericardial fluid to treat heart disease.

An embodiment comprises a composition comprising any composition or combination of compositions described herein.

An embodiment comprises a composition made by dissolving HA crosslinking partners in solvent such as an aqueous buffer. The HA crosslinking partners may be subsets of HA that have, naturally or by modification, complementary crosslinking reactive groups. The solvent may have a pH of 8.0. The pH may be 1, 2, 3, 4, 5, 6, 7, 8, 9, 10, 11, 12, 13, or 14, or between any two of 1, 2, 3, 4, 5, 6, 7, 8, 9, 10, 11, 12, 13, 14. The concentration of HA may be 1 mg/mL for each HA partner, giving a total HA concentration of 2 mg/mL. Embodiments include total HA concentrations greater than 1 microgram/mL, 10 micrograms/mL, 100 micrograms/mL, 1 mg/mL, 2 mg/mL, 5 mg/mL, 10 mg/mL, 20 mg/mL or 40 mg/mL, or between any of two of 1 microgram/mL, 10 micrograms/mL, 100 micrograms/mL, 1 mg/mL, 2 mg/mL, 5 mg/mL, 10 mg/mL, 20 mg/mL or 40 mg/mL, or any of 1 microgram/mL, 10 micrograms/mL, 100 micrograms/mL, 1 mg/mL, 2 mg/mL, 5 mg/mL, 10 mg/mL, 20 mg/mL or 40 mg/mL. A precipitating solvent is then added to cause precipitation of the HA crosslinking partners and enhancing the rate of chemical crosslinking. Alternatively, the HA solution may be added to a precipitating solvent to cause precipitation. The HA crosslinking partners may have crosslinking conversion of more than 1% prior to precipitation or partial conversion of crosslinking bonds may occur prior to precipitation. Pre-precipitation crosslinking conversion may be 1%, 10%, 25%, 50% or 75%. Secondary crosslinking reactions can be utilized after precipitation. The precipitating solvent may be an alcohol. The alcohol may be ethanol. The alcohol may be methanol or isopropanol. The precipitating solvent may be acetone. The final concentration of precipitating solvent may be 66% v/v. The final concentration of precipitating solvent may be 25% v/v, 30% v/v, 35% v/v, 40% v/v, 45% v/v, 50% v/v, 55% v/v, 60% v/v, 65% v/v, 70% v/v, 75% v/v, 80% v/v, 85% v/v, 90% v/v, 95% v/v, or greater than 99% v/v. Salts may be added to the precipitating solvent or HA solution to facilitate or cause precipitation and crosslinking of the HA. The salt may be sodium chloride or sodium acetate. Final concentrations of salt in the precipitating HA mixture may be greater than 0.1% w/v, 0.2% w/v, 0.3% w/v, 0.4% w/v, 0.5% w/v, 0.6% w/v, 0.7% w/v, 0.8% w/v, 0.9% w/v, 1% w/v, 2% w/v, 3% w/v, 4% w/v or 5% w/v, between any of 0.1% w/v, 0.2% w/v, 0.3% w/v, 0.4% w/v, 0.5% w/v, 0.6% w/v, 0.7% w/v, 0.8% w/v, 0.9% w/v, 1% w/v, 2% w/v, 3% w/v, 4% w/v or 5% w/v, or including any of 0.1% w/v, 0.2% w/v, 0.3% w/v, 0.4% w/v, 0.5% w/v, 0.6% w/v, 0.7% w/v, 0.8% w/v, 0.9% w/v, 1% w/v, 2% w/v, 3% w/v, 4% w/v or 5% w/v.

An embodiment comprises a microparticle composition wherein microparticle size is controlled during the precipitation process. An embodiment includes milling or sizing the precipitate to control microparticle size. Microparticle diameters in embodiments herein may include 10 microns, 35 microns, 50 microns, 75 microns, 100 microns, 200 microns, 300 microns, 400 microns, 500 microns, 600 microns, 700 microns, 800 microns, 900 microns, 1 mm, 2 mm after reconstitution in physiologic buffer. Microparticle sizes in embodiments herein may include a range of sizes between any two of 10 microns, 35 microns, 50 microns, 75 microns, 100 microns, 200 microns, 300 microns, 400 microns, 500 microns, 600 microns, 700 microns, 800 microns, 900 microns, 1 mm, 2 mm after reconstitution in physiologic buffer.

An embodiment comprises a composition made by dissolving chemically modified HA in solvent followed by precipitation and crosslinking of modified reactive groups. The solvent may have a pH of 7.0. The HA concentration may be 0.2% w/w. The precipitating solvent may be an alcohol. The alcohol may be ethanol. The final concentration of precipitating solvent may be 75% v/v. The reactive groups may be reacted following precipitation through changes in temperature, pH, time, light exposure or through the addition of chemical initiators or crosslinking molecules. Alternatively, partial conversion of reactive groups may occur prior to precipitation. Pre-precipitation crosslinking conversion may be 1%, 10%, 25% or 50%.

The product may be soft upon reconstitution in aqueous buffer. The product may be soft to such a degree that it has a storage modulus of less than 10 kPa. The product may be soft to such a degree that it has a storage modulus of less than 2 kPa. The product may be soft to such a degree that it has a storage modulus of less than 1 kPa. The product may be soft to such a degree that it has a storage modulus of between 10 Pa and 10 kPa. The product may be used in applications where a softer HA is desired. For example in lubricating applications such as the synovial fluid or pericardial fluid. A composition comprising the networks of hyaluronic acid (HA) made by this method are contemplated. The composition may comprise multiple fractions of HA. Each fraction may be made by separate iterations of this method. Different fractions may comprise networks of HA may be one or more iteration of this method combined with networks of HA made by another method(s). The other method(s) may be one or more method herein, or any HA crosslinking method.

The product may be optically transparent upon reconstitution in aqueous buffer. The product may be optically opaque upon reconstitution in aqueous buffer. The opacity of the product upon reconstitution in aqueous buffer may correlate with the crosslink density. Higher crosslink densities may maintain the precipitated form, while lower crosslinked densities may allow hydration and swelling leading to a semi-transparent product. The conformation of HA that has been crosslinked may be referred to as a collapsed conformation.

An embodiment comprises a network of HA that is crosslinked with chemistries containing hydrolytically liable bonds to control HA hydration and swelling with crosslinking bond hydrolysis after injection into the body.

An embodiment comprises a network of HA that comprises stabilized HA precipitates. An embodiment comprises a network of HA that are crosslinked precipitates of chemically modified hyaluronic acid.

An embodiment comprises a network of HA in the form of a partially swollen gel. The gel may be swollen with water or aqueous buffer. An embodiment comprises a network of HA in the form of a hydrogel. An embodiment comprises a network of HA in the form of microparticles. An embodiment comprises a network of HA in the form of nanoparticles. Nanoparticles may have an average diameter of greater than 1 nm, 10 nm, or 100 nm, or between any two of 1 nm, 10 nm, 100 nm or 1 micron, or any of 1 nm, 10 nm, 100 nm or 1 micron.

An embodiment comprises a composition comprising any one or more network herein. The compositions may comprise two or more separate HA networks. The two or more networks may hydrate and/or swell at different rates. An HA network herein may swell and uptake 2, 5, 10, 20, 50, 100, or 200 times its dry mass with water 24 hrs after being reconstituted with excess aqueous buffer. An HA network herein may swell and uptake between any two of 2, 5, 10, 20, 50, 100, or 200 times its dry mass with water 24 hrs after being reconstituted with excess aqueous buffer. Water uptake may be measured by centrifuging the material suspended in aqueous buffer, removing the supernatant and weighing the hydrated pellet.

An embodiment comprises a method of precipitating HA prior to or during crosslinking to limit hydration. The limit in hydration may limit swelling.

An embodiment comprises a method of treatment. The method comprises administering a composition herein to a subject in need thereof. The administering may be by injection to a site if interest. The injection may be via syringe. The injection may be via a catheter. The site of interest may be synovial fluid, pericardial fluid, blood plasma, topical wound bed, diabetic ulcer bed, tumor microenvironment, vitreous humor, surgical intervention site, vaginal canal, or cerebrospinal fluid. The subject may be human. The subject may be a dog, horse or cat. The subject may be a mammal. The method may comprise administering a composition herein comprising a high density HA network(s) to the body via injection. The injected high density HA network(s) may provide mechanical support from swelling HA as the crosslinks hydrolyze.

Any embodiment comprises a formulation comprising at least one of any precipitate, network or composition disclosed herein.

Embodiments include any method of making a precipitate, network, or composition disclosed herein.

An embodiment comprises any method of treatment disclosed herein.

The HA molecular weights in embodiments herein may be greater than 80 KDa, 100KDa, 200 KDa, 500 KDa, 700KDa, 1000 KDa, 2000 KDa, 3000 KDa, or 4000 KDa, in a range between and including any two of 80 KDa, 100 KDa, 200 KDa, 500 KDa, 700 KDa, 1000 KDa, 2000 KDa, 3000 KDa, or 4000 KDa, or one of 80 KDa, 200 KDa, 500 KDa, 700KDa, 1000 KDa, 2000 KDa, 3000 KDa, or 4000 KDa.

Networks of HA in embodiments herein may have a concentration of HA after reconstitution in physiological buffer greater than 10 mg/mL, 20 mg/mL, 30 mg/mL, 40 mg/mL, 50 mg/mL, 60 mg/mL, 70 mg/mL, 80 mg/mL, 90 mg/mL or 100 mg/mL, in a range between and including any two of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, or 200 mg/mL, or one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, or 200 mg/mL.

HA in embodiments herein may be in its native conformation. HA in its native conformation may readily swell with water and be optically clear. HA in embodiments herein may be in a precipitated or collapsed form. A precipitated or collapsed form of HA may not readily swell with water and be optically opaque.

Embodiments herein may include or be the result of crosslinking HA with crosslinkers containing hydrolytically degradable chemistries. The hydrolytically degradable chemistries may be selected from one or more of lactic acid, poly-L-lactic acid, caprolactone, polycaprolactone, glycolic acid, polyglycolic acid, hydroxyethyl methacrylate, thioesters, and anhydrides.

An extent of chemical crosslinking for an HA herein may be greater than 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, or 15%, in a range between and including any two of 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, 15% or 20%, or one of 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, 15% or 20%.

The HA in any embodiment herein may include chemically modified HA. The chemical modification may comprise at least one of an aldehyde, hydrazide, thiol, acrylate, methacrylate, hydroxyethylmethacrylate, norbornene, azide, alkyne, glycidyl methacrylate, haloacetate, benzyl ester, tyramide, glycidyl ether, epoxide, cyclodextrin, adamantane, or hydrolytically degradable moieties. The hydrolytically degradable moieties may be selected from one or more of lactic acid, poly-L-lactic acid, caprolactone, polycaprolactone, glycolic acid, polyglycolic acid, hydroxyethyl methacrylate, thioester and anhydrides.

HA in embodiments herein may include an extent of chemical modification greater than 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, or 15%, in a range between and including any two of 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, 15% or 20%, or one of 0.01%, 0.05%, 0.1%, 0.2%, 0.35%, 0.5%, 1%, 2%, 3%, 4%, 5%, 6%, 7%, 8%, 9%, 10%, 11%, 12%, 13%, 14%, 15% or 20%.

Particles in embodiments herein may be at sizes greater than 100 nm, 1 μm, 10 μm, 50 μm, 100 μm, 500 μm, or 1 mm in a range between and including any two of 100 nm, 1 μm, 10 μm, 50 μm, 100 μm, 500 μm, 1 mm, or 10 mm or any one of 100 nm, 1 μm, 10 μm, 50 μm, 100 μm, 500 μm, 1 mm, or 10 mm.

HA networks in embodiments herein may solubilize in phosphate buffered saline at 37° C., pH 7.4 over times greater than 2, 3, 4, 5, 6, 7, 8, 9, 10, 20, 30, 40, 50, 60, 70, 80, 90 or 180 days, in a range between and including any two of 2, 3, 4, 5, 6, 7, 8, 9, 10, 20, 30, 40, 50, 60, 70, 80, 90 or 180 days or any one of 2, 3, 4, 5, 6, 7, 8, 9, 10, 20, 30, 40, 50, 60, 70, 80, 90, or 180 days. In one aspect, one fraction solubilizes in 2 to 7 days; a second fraction solubilizes in 10 to 14 days; and a third fraction solubilizes in 17 to 21 days to mimic a three-injection regime. In and embodiment, one fraction solubilizes in 2 to 7 days; a second fraction solubilizes in 10 to 14 days; a third fraction solubilizes in 17 to 21 days; a fourth fraction solubilizes in 24 to 28 days and a fifth fraction solubilizes in 31 to 35 days to mimic a five-injection regime. In an embodiment, one fraction solubilizes with two weeks; a second fraction solubilizes within four weeks; and a third fraction solubilizes with 6 weeks. In another aspect, one fraction solubilizes within one month; a second fraction solubilizes within two months; and a third fraction solubilizes over three months. In an embodiment, one fraction solubilizes within one month; a second fraction solubilizes within two months; and a third fraction solubilizes over three months.

A composition herein may further comprise at least one hyaluronidase inhibitor. The hyaluronidase inhibitor r may prevent hyaluronidase mediated degradation of one or more of the HA fractions. The at least one hyaluronidase inhibitor may be separate from or embedded in one or more of the fractions. The hyaluronidase inhibitor(s) may be selected from sulfated polysaccharides. The sulfated polysaccharides may be but are not limited to heparin, heparan sulfate, chondroitin sulfate, and synthetically derived molecules. The synthetically derived molecules may be but are not limited to sulfated hyaluronic acid, dextran sulfate, and pentosan polysulfate. The sulfated polysaccharides may be partially or fully sulfated. The partially or fully sulfated polysaccharide described herein can be the pharmaceutically acceptable salt or ester thereof.

HA networks in embodiments herein may maintain exogenous HA at concentrations greater than 100 μg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days or any one of 2, 3, 4, 5, 6, 7, 8, 9, 10, 20, 30, 40, 50, 60, 70, 80, 90, or 180 days.

HA networks in embodiments herein may maintain exogenous HA at concentrations greater than 1 mg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days or any one of 2, 3, 4, 5, 6, 7, 8, 9, 10, 20, 30, 40, 50, 60, 70, 80, 90, or 180 days.

HA networks in embodiments herein may maintain exogenous HA at concentrations greater than 100 ug/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days or any one of 2, 3, 4, 5, 6, 7, 8, 9, 10, 20, 30, 40, 50, 60, 70, 80, 90, or 180 days.

HA networks in embodiments herein may maintain exogenous HA at concentrations greater than 10 mg/mL in fluids and tissues in the body over times greater than 2, 3, 4, 5, 6, 7, 8, 9, 10, 20, 30, 40, 50, 60, 70, 80, 90, or 180 days, in a range between and including any two of 2, 3, 4, 5, 6, 7, 8, 9, 10, 20, 30, 40, 50, 60, 70, 80, 90, or 180 days or any one of 2, 3, 4, 5, 6, 7, 8, 9, 10, 20, 30, 40, 50, 60, 70, 80, 90, or 180 days.

HA networks in embodiments herein may solubilize to increase the storage modulus of surrounding fluids by a value greater than 1 Pa, 10 Pa, 20 Pa, 30 Pa, 40 Pa, 50 Pa or 100 Pa upon solubilization, in a range between and including any two of 1 Pa, 10 Pa, 20 Pa, 30 Pa, 40 Pa, 50 Pa, 100 Pa, or 500 Pa upon solubilization, or any one of 1 Pa, 10 Pa, 20 Pa, 30 Pa, 40 Pa, 50 Pa, 100 Pa, or 500 Pa upon solubilization.

HA networks in embodiments herein may solubilize to increase the loss modulus of surrounding fluids by a value greater than 1 Pa, 10 Pa, 20 Pa, 30 Pa, 40 Pa, 50 Pa or 100 Pa upon solubilization, in a range between and including any two of 1 Pa, 10 Pa, 20 Pa, 30 Pa, 40 Pa, 50 Pa, 100 Pa, or 500 Pa upon solubilization, or any one of 1 Pa, 10 Pa, 20 Pa, 30 Pa, 40 Pa, 50 Pa, 100 Pa, or 500 Pa upon solubilization.

HA networks in embodiments herein may solubilize to increase the viscosity of surrounding fluids by a value greater than 1 Pa, 10 Pa, 20 Pa, 30 Pa, 40 Pa, 50 Pa, 100 Pa, or 500 Pa upon solubilization, in a range between and including any two of 1 Pa, 10 Pa, 20 Pa, 30 Pa, 40 Pa, 50 Pa, 100 Pa, or 500 Pa upon solubilization, or any one of 1 Pa, 10 Pa, 20 Pa, 30 Pa, 40 Pa, 50 Pa, 100 Pa, or 500 Pa upon solubilization. The embodiments may include formulations comprising the HA networks.

Embodiments may include formulations comprising one or more HA network herein.

Embodiments include formulations comprising more than one HA network or fraction that solubilize at different rates. Embodiments include formulations with 2, 3, 4, 5, 6, 7, 8, 9 or 10 separate networks or fractions.

An embodiment comprises a method of HA network fabrication in which HA is first precipitated in a nonaqueous solvent known in the art to precipitate HA including but not limited to ethanol, acetone, or isopropanol. An embodiment includes adding the solvent to a solution of HA. An embodiment includes adding a solution of HA to the solvent. Embodiments include aqueous and non-aqueous solutions of HA and salts of HA including but not limited to sodium and tetrabutyl ammonium salts. An embodiment comprises a method of HA network fabrication in which HA is first precipitated under conditions that cause precipitation. The conditions that cause precipitation may be, but are not limited to, salt concentration, pH, and temperature. An embodiment comprises a method of crosslinking the precipitated HA in which the HA is chemically modified and reactive groups crosslink within the precipitate. Embodiments include precipitating two chemically modified HAs with complimentary reactive groups wherein the reactive groups react within the precipitate during or after the precipitation process. Embodiments include adding initiators, crosslinking molecules, heat or light after precipitating the chemically modified HA to crosslink and stabilize the precipitates. Embodiments include using combinations of polar and non-polar solvents to facilitate crosslinking chemical reactions. Embodiments including using dimethylsulfoxide to facilitate crosslinking reactions. Embodiments include using dimethylsulfoxide and alcohol mixtures. Embodiments include chemical reactions involving free radical generation and propagation.

Embodiments include precipitating aqueous solution of HA and non-aqueous solutions of HA. Non-aqueous solvents may be used including but not limited dimethylsulfoxide and dimethylformamide. Salts of HA including but not limited to HA-tetrabutylammonium may be used to produce non-aqueous solutions of HA.

A composition herein may comprise a drug. The drug may be selected from small molecules, peptides, cytokines, proteins, polysaccharides, synthetic polymers, particles, DNA plasmids, mRNA, cells, cellular exosomes and other cellular components.

An embodiment comprises a method of HA network fabrication and drug encapsulation in which HA and drug is first precipitated in a nonaqueous solvent known in the art to precipitate HA and drug including but not limited to ethanol, acetone, or isopropanol. An embodiment comprises a method of HA network fabrication and drug encapsulation in which HA is first precipitated under conditions that cause precipitation. The conditions that cause precipitation may be, but are not limited to, salt concentration and temperature.

An embodiment comprises a method of HA network fabrication and protein encapsulation in which HA and protein is first precipitated in a nonaqueous solvent known in the art to precipitate HA and protein including but not limited to ethanol, acetone, or isopropanol. An embodiment comprises a method of HA network fabrication and protein encapsulation in which HA is first precipitated under conditions that cause precipitation. The conditions that cause precipitation may be, but are not limited to, salt concentration, pH and temperature.

A composition herein may comprise a pharmaceutically acceptable carrier. Embodiments include carriers of uncrosslinked or crosslinked HA in aqueous buffer. In an embodiment, the HA carrier concentration is greater than 1 mg/mL. Embodiments include HA carrier concentrations between 10, 20, 40 and 80 mg/mL. Embodiments include HA precipitates encapsulated in crosslinked HA hydrogels.

Any one or more composition herein may be used in a method of treating inflammation. Any one or more composition herein may be used in a method of treating osteoarthritis. Any one or more composition herein may be used in a method of treating ocular disease. Any one or more composition herein may be used in a method of treating cardiovascular disease. Any one or more composition herein may be used in a method of treating disc degeneration. Any one or more composition herein may be used in a method of treating diabetic ulcers. Any one or more composition herein may be used in a method of treating pain. Any one or more composition herein may be used in a method of reducing inflammation following surgery. Any one or more composition herein may be used in a method to improve tissue healing following surgery or a traumatic injury. A method of treating may comprise administering one or more composition herein to one of the foregoing sites. The administering may be by injection. The injection may be via syringe, or via catheter.

An embodiment comprises the use of any composition herein for treating inflammation. An embodiment comprises the use of any composition herein for treating osteoarthritis. An embodiment comprises the use of any composition herein for treating ocular disease. An embodiment comprises the use of any composition herein for treating cardiovascular disease. An embodiment comprises the use of any composition herein for treating disc degeneration. An embodiment comprises the use of any composition herein for treating diabetic ulcers. An embodiment comprises the use of any composition herein for treating pain. An embodiment comprises the use of any composition herein for reducing inflammation following surgery. An embodiment comprises the use of any composition herein for improving tissue healing following surgery or a traumatic injury.

An embodiment comprises the use of two or more compositions herein in combination for treating inflammation. An embodiment comprises the use of two or more compositions herein in combination for treating osteoarthritis. An embodiment comprises the use of two or more compositions herein in combination for treating ocular disease. An embodiment comprises the use of two or more compositions herein in combination for treating cardiovascular disease. An embodiment comprises the use of two or more compositions herein in combination for treating disc degeneration. An embodiment comprises the use of two or more compositions herein in combination for treating diabetic ulcers. An embodiment comprises the use of two or more compositions herein in combination for treating pain. An embodiment comprises the use of two or more compositions herein in combination for reducing inflammation following surgery. An embodiment comprises the use of two or more compositions herein in combination for improving tissue healing following surgery or a traumatic injury.

Any one or more formulation herein may be used in a method of treating inflammation. Any one or more formulation herein may be used in a method of treating osteoarthritis. Any one or more formulation herein may be used in a method of treating ocular disease. Any one or more formulation herein may be used in a method of treating cardiovascular disease. Any one or more formulation herein may be used in a method of treating disc degeneration. Any one or more formulation herein may be used in a method of treating diabetic ulcers. Any one or more formulation herein may be used in a method of treating pain. Any one or more formulation herein may be used in a method of reducing inflammation following surgery. Any one or more formulation herein may be used in a method to improve tissue healing following surgery or a traumatic injury. A method of treating may comprise administering one or more formulation herein to one of the foregoing sites. The administering may be by injection. The injection may be via syringe, or via catheter.

An embodiment comprises the use of any formulation herein for treating inflammation. An embodiment comprises the use of any formulation herein for treating osteoarthritis. An embodiment comprises the use of any formulation herein for treating ocular disease. An embodiment comprises the use of any formulation herein for treating cardiovascular disease. An embodiment comprises the use of any formulation herein for treating disc degeneration. An embodiment comprises the use of any formulation herein for treating diabetic ulcers. An embodiment comprises the use of any formulation herein for treating pain. An embodiment comprises the use of any formulation herein for reducing inflammation following surgery. An embodiment comprises the use of any formulation herein for improving tissue healing following surgery or a traumatic injury.

An embodiment comprises the use of two or more formulations herein in combination for treating inflammation. An embodiment comprises the use of two or more formulations herein in combination for treating osteoarthritis. An embodiment comprises the use of two or more formulations herein in combination for treating ocular disease. An embodiment comprises the use of two or more formulations herein in combination for treating cardiovascular disease. An embodiment comprises the use of two or more formulations herein in combination for treating disc degeneration. An embodiment comprises the use of two or more formulations herein in combination for treating diabetic ulcers. An embodiment comprises the use of two or more formulations herein in combination for treating pain. An embodiment comprises the use of two or more formulations herein in combination for reducing inflammation following surgery. An embodiment comprises the use of two or more formulations herein in combination for improving tissue healing following surgery or a traumatic injury.

### Embodiments List

The following list of embodiments does not limit the embodiments otherwise described herein.
1. A composition comprising a crosslinked precipitate of chemically modified hyaluronic acid.
2. The composition of embodiment 1, wherein the crosslinked precipitate comprises hydrolytically degradable crosslinks.
3. The composition of embodiment 1 or 2, wherein the crosslinked precipitate comprises crosslinks at an extent of chemical crosslinking less than 2%.
4. The composition of any of embodiments 1-3, wherein the chemically modified hyaluronic acid comprises at least one of acrylate or methacrylate chemical modification.
5. The composition of any of embodiments 1-4, wherein the crosslinked precipitate is formed by introducing an aqueous solution of chemically modified HA to a solvent.
6. The composition of any of embodiments 1-5, wherein the crosslinked precipitate is optically opaque.
7. The composition of any of embodiments 1-6, wherein the crosslinked precipitate forms microparticles between 10 microns and 1 millimeter in diameter when reconstituted in aqueous buffer.
8. The composition of any of embodiments 1-7, wherein the crosslinked precipitate comprises more than one fraction of stabilized precipitates of hyaluronic acid, and the fractions solubilize at different rates.
9 The composition of any of embodiments 1, 2, and 4-8, wherein the crosslinked precipitate comprises crosslinks at an extent of chemical crosslinking less than 10%.
10. The composition of any of embodiments 1, 2, and 4-8, wherein the crosslinked precipitate comprises crosslinks at an extent of chemical crosslinking less than 5%.
11. The composition of any of embodiments 1, 2, and 4-8, wherein the precipitate comprises an extent of hyaluronic acid crosslinking, and the extent is less than 1%.
12. A method of making a network of hyaluronic acid comprising precipitating a first chemically modified hyaluronic acid to facilitate crosslinking.
13. The method of embodiment 12 further comprising precipitating a second chemically modified hyaluronic acid with the first chemically modified hyaluronic acid, wherein the second chemically modified hyaluronic acid comprises second chemical modifications, the first chemical modified hyaluronic acid comprises first chemical modifications, and the first and second chemical modifications are complementary.
14. The method of embodiment 14, wherein the second chemical modifications and the first chemical modifications are independently selected from a methacrylate group, a thiol group or an acrylate group.
15. The method of any of embodiments 12-14 further comprising adding chemical initiators to the precipitated hyaluronic acid to initiate crosslinking.
16. The method of any of embodiments 12-15 further comprising applying at least one of light or heat to the precipitated hyaluronic acid to initiate crosslinking following precipitation.
17. The method of embodiment 12, wherein the first chemically modified hyaluronic acid comprises first chemical modifications and the first chemical modifications comprise a methacrylate group or an acrylate group.
18. A method of treatment comprising injecting into a body of a subject the compositions of any one of embodiments 1-11 or the product of any one of embodiments 12-17 reconstituted in aqueous buffer.
19. The method of embodiment 18, wherein the subject is a mammal.
20. The method of embodiment 19, wherein the mammal is a human.
21. The method of any of embodiments 18-20, wherein the injecting into a body of the subject comprises injecting into synovial fluid of the subject to treat osteoarthritis.
22. The method of any of embodiments 18-20, wherein the injecting into a body of the subject comprises injecting into pericardial fluid of the subject to treat heart disease.

### EXAMPLES

### Example 1

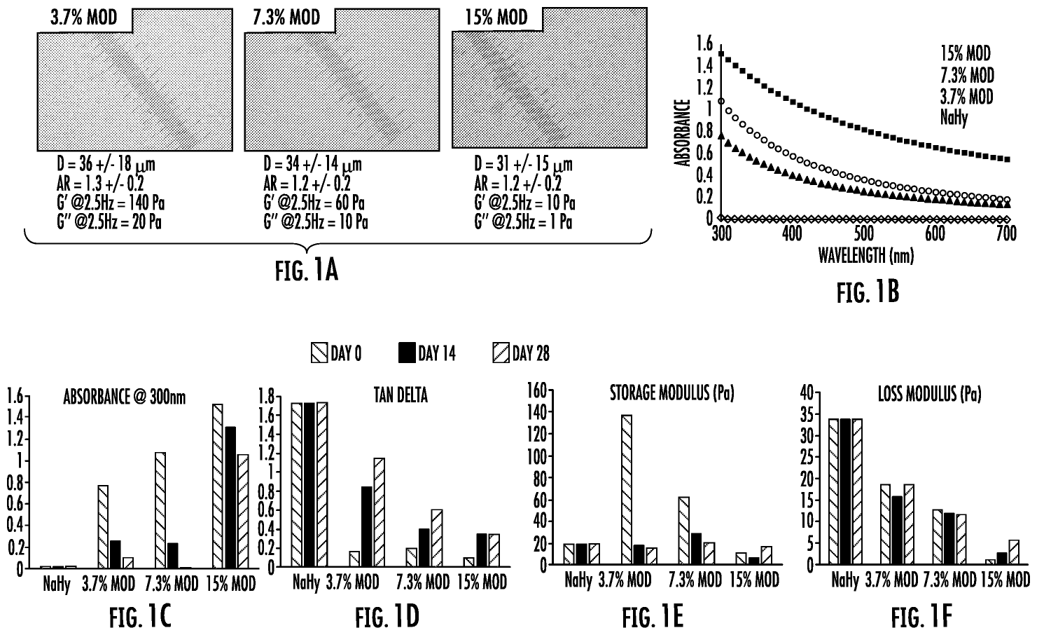

Sodium hyaluronate (700 kDa, Lifecore Biomedical) was first converted to a tetrabutylammonium (TBA) salt and then chemically modified with hydroxyethylmethacrylate through an esterification reaction in dimethylsulfoxide. The chemically modified HA was then purified and dried. The percent of disaccharides chemically modified with hydroxyethylmethacrylate was determined with 1H NMR (Bruker 600 MHz). The hydroxyethylmethacrylate modified HA was then converted to a thiol modified HA through reaction with excess dithiothreitol (20:1 thiol: methacrylate, 3 days at room temperature) and purified. Complete conversion was confirmed with the loss of methacrylate peaks on 1H NMR spectra. Thiol modified HA and hydroxyethylmethacrylate modified HA with the same percent modifications were dissolved in water at 2 mg/mL and then mixed at 1:1 mass ratios. NaCl was added to the solution at a final concentration of 0.24 M and then a 3× volume of ethanol was dripped into the HA solution with stirring to precipitate. The precipitate was allowed to stir overnight at room temperature. The precipitate was washed with a 1:4 water: ethanol mixture, followed by an ethanol wash before drying and milling into a powder. Precipitated and crosslinked powders fabricated with 3.7, 7.3 and 15% hydroxyethylmethacrylate modified HA were reconstituted with PBS, stained with dimethylmethylene blue and imaged with optical microscopy to measure particles dimensions (FIG. 1A). The storage (G′) and loss (G″) moduli of 2 wt % particle suspensions in PBS were measured with oscillatory rheology (TA Instruments HR2, 20 mm 1 degree cone and plate, 28 micron gap) (FIG. 1A). The 2 wt % particle suspensions formed a white opaque material. Opacity was measured with absorbance on a plate reader (Biotek) (FIG. 1B). Particle suspensions were incubated in PBS at 37C on an orbital shaker set to 80 rpm and opacity and rheological properties were measured over time (FIGS. 1C-1F). The particles became more transparent over time and the rheological properties exhibited a shift from gel to viscous HA materials as the particles solubilized.

### Example 2

Sodium hyaluronate (700 kDa, Lifecore Biomedical) was first converted to a tetrabutylammonium (TBA) salt and then chemically modified with hydroxyethylmethacrylate through an esterification reaction in dimethylsulfoxide. The chemically modified HA was then purified and dried.

The percent of disaccharides chemically modified with hydroxyethylmethacrylate was determined with 1H NMR (Bruker 600 MHz). Hydroxyethylmethacrylate modified HA with 3.7% extent of chemical modification was dissolved in aqueous buffer at 2 mg/mL and 3M NaCl was added at 5% v/v before dripping in 300% v/v ethanol to precipitate the HA. Ammonium persulfate (APS) and tetramethylethylenediamine (TEMED) were added to the precipitated HA suspension at final concentrations of 100 mM. The suspensions were allowed to stir overnight at room temperature. The reactions were then centrifuged at 4000 rpm for 5 min and the pellet was washed twice with ethanol and then vacuum dried. Powders were reconstituted at 20 mg/mL in PBS. The viscoelastic properties of the 20 mg/mL precipitate crosslinked with 100 mM APS and TEMED were as follows: G′@2.5 Hz=20 Pa, G″@2.5 Hz=1.5 Pa, tan delta @ 2.5 Hz=0.075 (TA Instruments HR2, 20 mm 1 degree cone and plate, 28 micron gap).

### Example 3

Sodium hyaluronate (700 kDa, Lifecore Biomedical) was first converted to a tetrabutylammonium (TBA) salt and then chemically modified with hydroxyethylmethacrylate through an esterification reaction in dimethylsulfoxide. The chemically modified HA was then purified and dried. The percent of disaccharides chemically modified with hydroxyethylmethacrylate was determined with 1H NMR (Bruker 600 MHz). Hydroxyethylmethacrylate modified HA with 3.7%, 7.3%, 10%, and 15% extent of chemical modification was dissolved in aqueous buffer at 2 mg/mL and 3M NaCl was added at 5% v/v before dripping in 300% v/v ethanol to precipitate the HA. The precipitate was allowed to settle, the supernatant was poured off and a 3:1 dimethylsulfoxide: aqueous buffer (0.1M phosphate, 50 mM triethanolamine, 0.15M sodium chloride, pH 8.5) was added to the precipitate. The suspension was then stirred and APS/TEMED were added at a final concentration of 25 mM and riboflavin phosphate was added at a final concentration of 50 micrograms per milliliter and the mixture was stirred overnight at room temperature. The crosslinked precipitates were then washed with ethanol and dried under vacuum. Crosslinked HA precipitates were reconstituted in PBS at 20 mg/mL and the opacity and rheological properties were measured. The 300 nm absorbance values were 1.35, 1.97, 2.03 and 2.02 for 3.7%, 7.3%, 10%, and 15% percent modifications, respectively. The tan delta values at 0.5 Hz, 1% strain were 0.14, 0.07, 0.05, and 0.06 for 3.7%, 7.3%, 10%, and 15% percent modifications, respectively. The storage moduli at 0.5 Hz, 1% strain were 274 Pa, 322 Pa, 911 Pa, and 337 Pa for 3.7%, 7.3%, 10%, and 15% percent modifications, respectively (TA Instruments HR2, 20 mm 1 degree cone and plate, 28 micron gap).

### Example 4

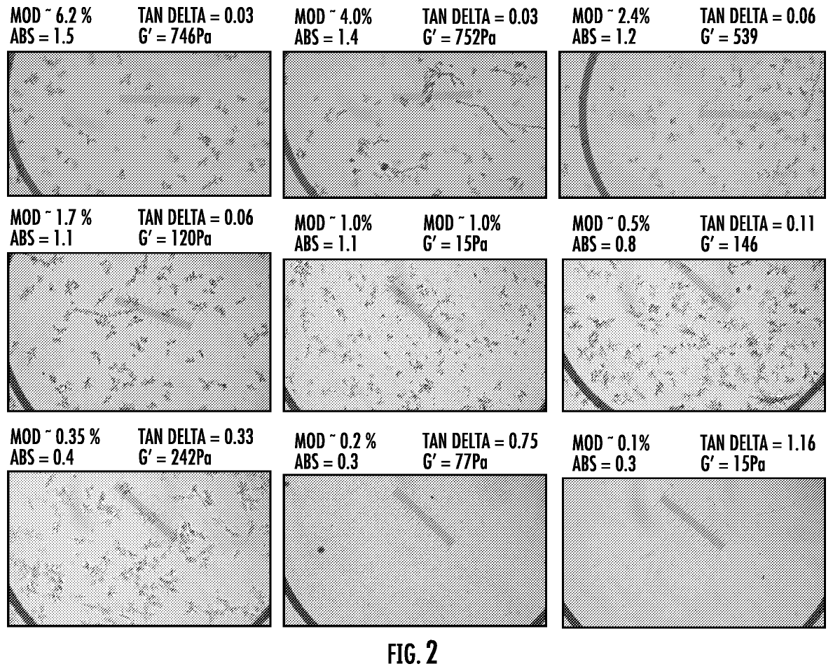

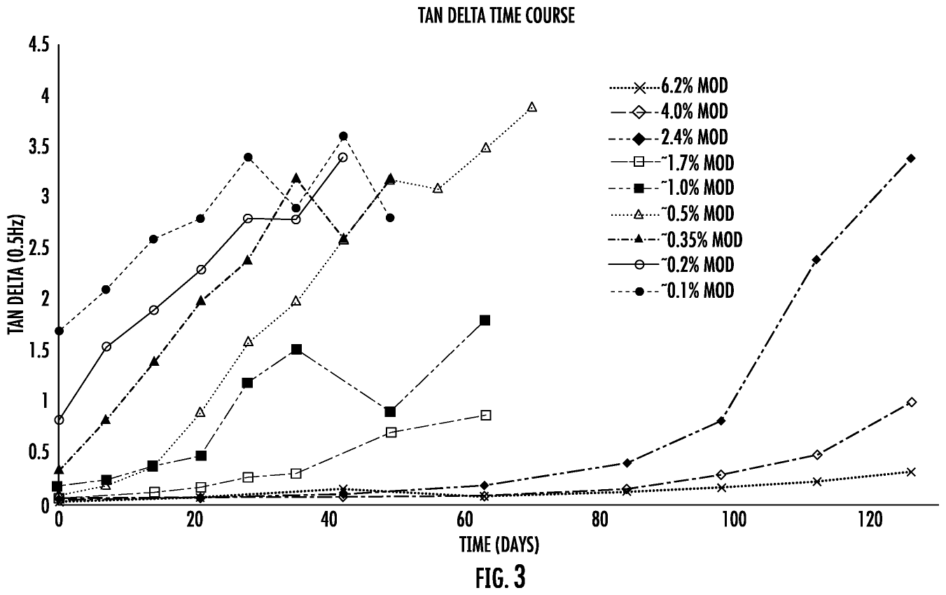

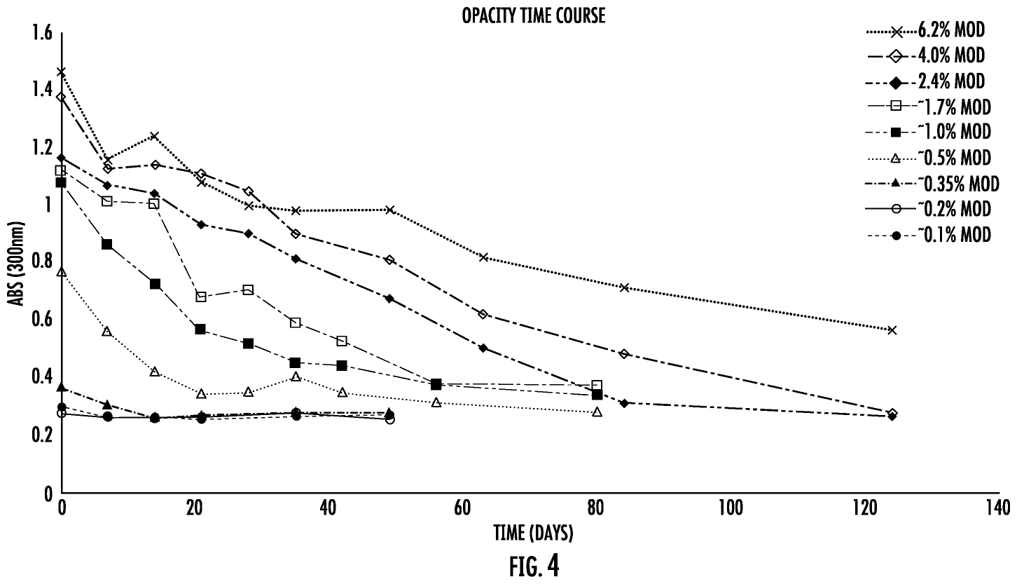

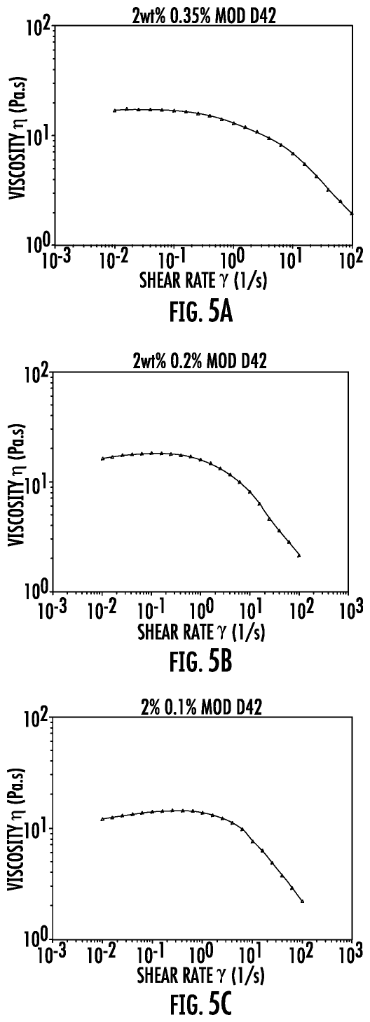

Sodium hyaluronate (700 kDa, Lifecore Biomedical) was chemically modified with an acrylate functional group by adding acrylic anhydride to an aqueous solution of sodium hyaluronate and maintaining the pH at 8.5 with NaOH for 18 hrs. The chemically modified HA was then purified and dried. The percent of disaccharides chemically modified with acrylate was determined with 1H NMR (Bruker 600 MHz). Percent modifications could be quantified down to 2% extent of chemical modification at which point the acrylate peaks became difficult to resolve. Lower modifications were approximated by extrapolating modifications from HA polymers with percent modification greater than 2% based on the amount of acrylic anhydride added. The acrylate modified HA was then converted to a thiol modified HA through reaction with excess dithiothreitol (20:1 thiol: methacrylate, 1 days at room temperature) and purified. Complete conversion was confirmed with the loss of acrylate peaks on 1H NMR spectra. Thiol modified HA and acrylate modified HA with the same percent modifications were dissolved in 25 mM triethanolamine buffer, pH 8.5 at 2 mg/mL and then mixed at 1:1 mass ratios. NaCl was added to the solution at a final concentration of 0.20 M and then a 2× volume of ethanol was dripped into the HA solution with stirring to precipitate. The precipitate was allowed to stir for 18 hrs at room temperature. The precipitate was then washed with a 1:4 water: ethanol mixture, followed by an ethanol wash before drying under vacuum. Precipitated and crosslinked powders were reconstituted with PBS, stained with dimethylmethylene blue and imaged with optical microscopy to measure particles dimensions (FIG. 2). Tan delta of 2 wt % particle suspensions in PBS were measured with oscillatory rheology (TA Instruments HR2, 20 mm 1 degree cone and plate, 28 micron gap, 0.5 Hz, 1% strain) (FIG. 2). Opacity was measured with absorbance on a plate reader (Biotek) (FIG. 2). Particle suspensions were incubated in PBS at 37C on an orbital shaker set to 80 rpm and opacity and rheological properties were measured over time (FIGS. 3 and 4). The particles became more transparent over time and the rheological properties exhibited a shift from gel to viscous HA materials as evidenced by an increase in tan delta as the particles solubilized. Following complete solubilization (clear solution with stabilized tan delta values), the rheological properties exhibited solutions of sodium hyaluronic (FIGS. 5A-5B).

### Example 5

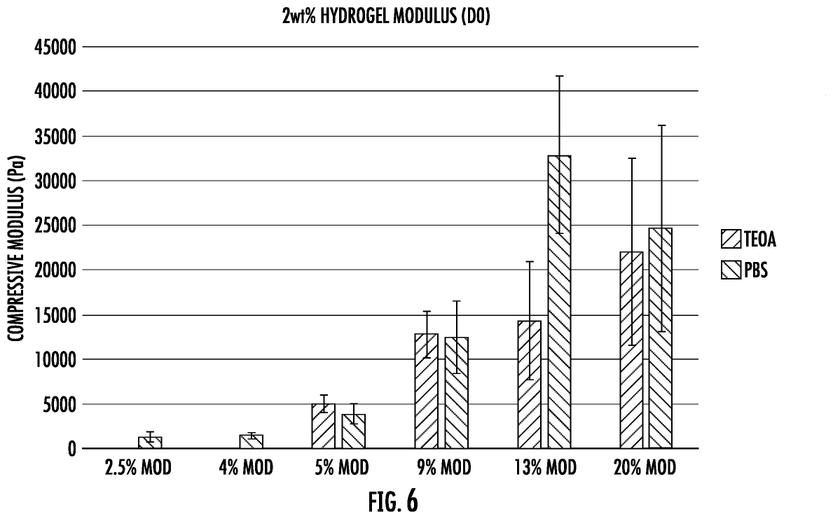

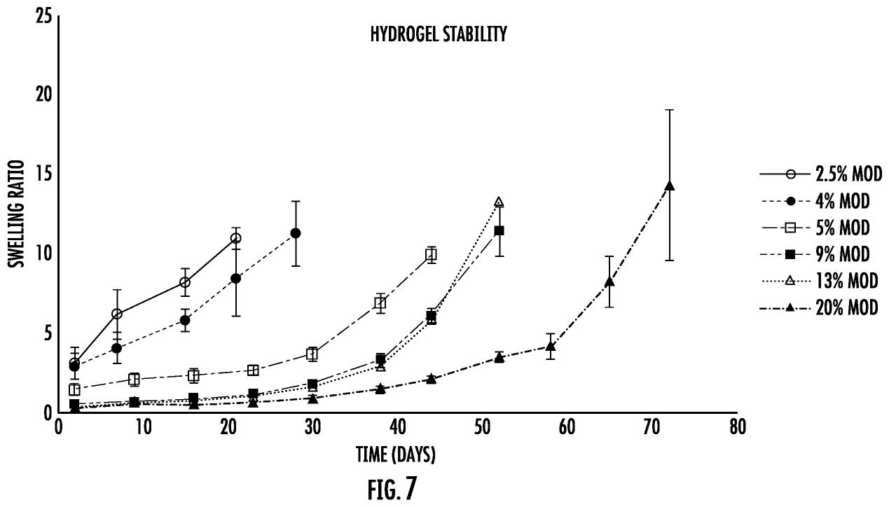

Sodium hyaluronate (700 kDa, Lifecore Biomedical) was chemically modified with an acrylate functional group by adding acrylic anhydride to an aqueous solution of sodium hyaluronate and maintaining the pH at 8.5 with NaOH for 18 hrs. The chemically modified HA was then purified and dried. The percent of disaccharides chemically modified with acrylate was determined with 1H NMR (Bruker 600 MHz). 2 wt % HA hydrogels were formed by mixing acrylated HA with dithiothreitol at a molar ratio of 1:1 acrylate: thiol in PBS or 0.1M phosphate buffer with 50 mM triethanolamine, adding the precursor solution to cylindrical molds and incubating at room temperature overnight. The compressive modulus of the gels were measured by applying a 20% strain at a constant strain rate (FIG. 6). 50 μL hydrogels crosslinked in PBS were incubated in 1 mL PBS and hydrogel masses were measured overtime and buffer collected and replaced with fresh PBS. Swelling ratios were calculated by subtracting the original hydrogel mass from the hydrogel mass and dividing by the original hydrogel mass. Hydrogel masses were measured and swelling ratios calculated until to gel could not be separated from the buffer indicating complete dissolution (FIG. 7).

### Example 6

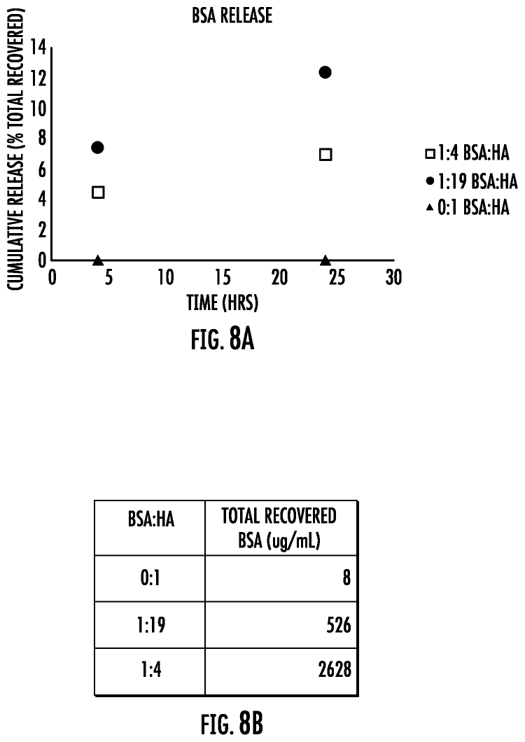

Sodium hyaluronate (700 kDa, Lifecore Biomedical) was chemically modified with an acrylate functional group by adding acrylic anhydride to an aqueous solution of sodium hyaluronate and maintaining the pH at 8.5 with NaOH for 18 hrs. The chemically modified HA was then purified and dried. Acrylated HA with extents of modification of approximately 1 was used to form precipitates. A portion of the acrylate modified HA was then converted to a thiol modified HA through reaction with excess dithiothreitol (20:1 thiol: methacrylate, 1 days at room temperature) and purified. Thiol modified HA and acrylate modified HA with the same percent modifications were dissolved in 25 mM triethanolamine buffer, pH 8.5 at 2 mg/mL and then mixed at 1:1 mass ratios. Bovine serum albumin (BSA) was added to the HA solution at 0:1, 1:19 and 1:4 BSA: HA mass ratio. NaCl was added to the solution at a final concentration of 0.20 M and then a 2× volume of ethanol was dripped into the HA solution with stirring to precipitate. The precipitate was allowed to stir for 18 hrs at room temperature. The precipitate was then washed with a 1:4 water: ethanol mixture, followed by an ethanol wash before drying under vacuum. Precipitated and crosslinked powders were reconstituted with PBS at 10 mg/mL and incubated at 37C. The particles were spun down after 4 and 24 hrs and supernatant collected and refreshed. After 24 hrs the particles were hydrolyzed in 25 mM NaOH. The protein content in the supernatant and hydrolyzed samples were analyzed with a BCA assay (Thermo Fisher) and shown in FIGS. 8A and 8B.

### Example 7

Sodium hyaluronate (700 kDa, Lifecore Biomedical) was chemically modified with an acrylate functional group by adding acrylic anhydride to an aqueous solution of sodium hyaluronate and maintaining the pH at 8.5 with NaOH for 18 hrs. The chemically modified HA was then purified and dried. Acrylated HA with extents of modification of approximately 1.7, 0.5 and 0.2 were used to form precipitates. A portion of the acrylate modified HA was then converted to a thiol modified HA through reaction with excess dithiothreitol (20:1 thiol: methacrylate, 1 days at room temperature) and purified. Thiol modified HA and acrylate modified HA with the same percent modifications were dissolved in 25 mM triethanolamine buffer, pH 8.5 at 2 mg/mL and then mixed at 1:1 mass ratios. Bovine serum albumin (BSA) was added to the HA solution at 1:1 mass ratio. NaCl was added to the solution at a final concentration of 0.20 M and then a 2× volume of ethanol was dripped into the HA solution with stirring to precipitate. The precipitate was allowed to stir for 18 hrs at room temperature. The precipitate was then washed with a 1:4 water: ethanol mixture, followed by an ethanol wash before drying under vacuum. Precipitated and crosslinked powders were reconstituted with PBS at 20 mg/mL and rheological properties were measured with oscillatory rheology (1 degree cone and plate geometry, 28 micron gap, 1% strain, 0.5 Hz, 37C). See Table 1.

TABLE 1

| | 1.7% mod | 0.5% mod | 0.2% mod |
|---|---|---|---|
| G′@0.5 Hz | 110 | 125 | 320 |
| G″@0.5 Hz | 13 | 29 | 45 |
| Tan delta 0.5 Hz | 0.14 | 0.23 | 0.14 |
| G′ @2.5 Hz | 142 | 162 | 369 |
| G″ @2.5 Hz | 20 | 50 | 55 |
| Tan delta 2.5 Hz | 0.14 | 0.31 | 0.15 |
| N0.1 | 169 | 99.8 | 959 |
| N1 | 12.7 | 8.7 | 141 |
| N10 | 1.7 | 1.1 | 10.3 |

### Example 8

Crosslinked precipitates of acrylated HA and DTT were fabricated as in Example 4 using acrylated HA with extents of chemical modification of 0.2 and 0.5. Formulations with 3.3 and 5.5 wt % (33 and 55 mg/mL) of HA precipitate particles were produced by reconstituting equal masses of the two fractions and unmodified sodium hyaluronate with PBS. The rheological properties were measured with oscillatory rheology (1 degree cone and plate geometry, 28 micron gap, 1% strain, 0.5 Hz, 37C, Table 2, n=2 per formulation).

TABLE 2

| | 3.3 wt % | 5.5 wt % |
|---|---|---|
| G′@0.5 Hz | 277 | 718 |
| G″@0.5 Hz | 223 | 526 |
| Tan delta 0.5 Hz | 0.83 | 0.74 |
| G′ @2.5 Hz | 597 | 1648 |
| G″ @2.5 Hz | 381 | 847 |
| Tan delta 2.5 Hz | 0.66 | 0.53 |
| N0.1 | 708 | 1737 |
| N1 | 185 | 315 |
| N10 | 38 | 63 |

### Example 9

Crosslinked precipitates of acrylated HA and DTT were fabricated as in Example 4 using acrylated HA with extents of chemical modification of 0.2, 0.5 and 1.7. Formulations with 2.2, 3.3 and 5.5 wt % (22, 33 and 55 mg/mL) of HA precipitate particles were produced by reconstituting equal masses of the three fractions with PBS. The rheological properties were measured with oscillatory rheology (1 degree cone and plate geometry, 28 micron gap, 1% strain, 0.5 Hz, 37C, Table 3, n=2 per formulation). 30 mg of each formulation was injected into the synovial cavity of rats using a 31G needle (n=3 per group) and knee swelling was measured over 1 week with calipers. No significant differences in knee swelling compared to 30 mg saline vehicle control was observed (n=3 per group, t-test compared to saline control with p<0.05 considered statistically significant).

TABLE 3

| | 2.2 wt % | 3.3 wt % | 5.5 wt % |
|---|---|---|---|
| G′@0.5 Hz | 105 | 203 | 1208 |
| G″@0.5 Hz | 30 | 72 | 434 |
| Tan delta 0.5 Hz | 0.29 | 0.36 | 0.37 |
| G′ @2.5 Hz | 140 | 293 | 1817 |
| G″ @2.5 Hz | 47 | 111 | 618 |
| Tan delta 2.5 Hz | 0.34 | 0.38 | 0.34 |
| N0.1 | 63 | 161 | 1102 |
| N1 | 13 | 30 | 176 |
| N10 | 3.8 | 9.8 | 40 |

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims: the above description: and/or shown in the attached drawings.

1. A crosslinked precipitate of hyaluronic acid, wherein the crosslinked precipitate comprises crosslinks that are hydrolytically degradable.

2. (canceled)

3. The crosslinked precipitate of claim 1, wherein the crosslinks are ester crosslinks.

4. (canceled)

5. The crosslinked precipitate of claim 1, wherein the crosslinked precipitate_is formed by combining an aqueous solution of a chemically modified hyaluronic acid with a solvent.

6. (canceled)

7. The crosslinked precipitate of claim 1, wherein the crosslinked precipitate forms microparticles between 10 microns and 1 millimeter in diameter when reconstituted in an aqueous buffer.

8. The crosslinked precipitate of claim 1, wherein the hyaluronic acid comprises more than one fraction of hyaluronic acid, and the fractions of hyaluronic acid solubilize at different rates.

9. (canceled)

10. The crosslinked precipitate of claim 1, an extent of crosslinking in the crosslinked precipitate is 0.01 to 5%.

11. (canceled)

12. A method of making the crosslinked precipitate of claim 1 comprising precipitating an aqueous solution of chemically modified hyaluronic acid with a solvent.

13. (canceled)

14. (canceled)

15. (canceled)

16. (canceled)

17. (canceled)

18. A method of treatment comprising injecting into a body of a subject the crosslinked precipitate of claim 1 reconstituted in aqueous buffer.

19. (canceled)

20. (canceled)

21. (canceled)

22. (canceled)

23. A use of the crosslinked precipitate of claim 1 reconstituted in aqueous buffer for at least one of_treating inflammation, treating osteoarthritis, treating ocular disease, treating cardiovascular disease, treating disc degeneration, treating diabetic ulcers, treating pain, reducing inflammation following surgery, or improving tissue healing following surgery or a traumatic injury.

24. A method of making an acrylate modified hyaluronic acid comprising:
adding acrylic anhydride to an aqueous solution of hyaluronic acid at a molar ratio of 0.002 to 1.5 of acrylic anhydride to hyaluronic acid monomer, wherein the pH is maintained between 6 and 10.

25. An acrylate modified hyaluronic acid, wherein an extent of acrylate chemical modification is 0.01 to 5 percent.

26. A chemically modified or crosslinked hyaluronic acid represented by Formula (I) as shown below, 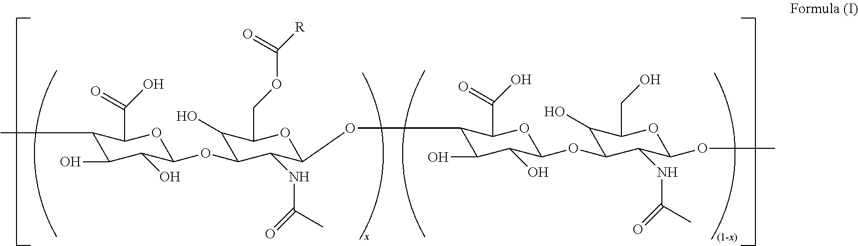
wherein an extent of chemical modification is 0.01 to 5.0 percent, or the extent of crosslinking is 0.01 to 5.0 percent.

27. The chemically modified hyaluronic acid of claim 26, wherein R is a vinyl group (—CH═CH₂).

28. The chemically modified or crosslinked hyaluronic acid of claim 26, wherein R is (—CH₂—CH₂—S—R′).

29. The chemically modified or crosslinked hyaluronic acid of claim 26 further comprising variations of Formula (I), wherein other groups on hyaluronic acid are chemically modified or crosslinked.

30. The crosslinked precipitate of claim 1, wherein the crosslinked precipitate is formed by precipitating an acrylate modified hyaluronic acid with multifunctional thiol molecules.

31. The crosslinked precipitate of claim 1 further comprising a peptide, polypeptide or protein

32. The crosslinked precipitate of claim 1, wherein the crosslinked precipitate solubilizes in less than 180 days when reconstituted in aqueous buffer at 10 mg/mL and incubated under physiological conditions.
